(19) United States
(12) Patent Application Publication
(10) Pub. No.: US 2025/0375586 A1
(43) Pub. Date: Dec. 11, 2025

(54) Gas Flow Monitoring Device

(71) Applicants: United States Government as Represented by the Department of Veterans Affairs, Washington, DC (US); University of Illinois-Chicago, Chicago, IL (US)

(72) Inventors: Israel Rubinstein, Chicago, IL (US); Braden Petno, Cleveland, OH (US)

(21) Appl. No.: 19/232,348

(22) Filed: Jun. 9, 2025

Related U.S. Application Data
(60) Provisional application No. 63/657,403, filed on Jun. 7, 2024.

(51) Int. Cl. A61M 16/06; A61B 5/06; A61M 16/00

(52) U.S. Cl. CPC A61M 16/0666; A61B 5/061; A61M 16/0003

(57) ABSTRACT

A device for determining the position of a nasal cannula is disclosed. The device includes nasal prongs coupled to a connecting tube. The nasal prongs are configured to insert into the nostrils of a user. Each nasal prong has a nasal prong electrode. The nasal prong electrodes are incorporated into a voltage divider which uses the skin resistance on the nasal prongs as an element in the divider thereby acting as an indicator of proper wear of the nasal cannula. A device configured to communicate with flex sensors of a nasal cannula and nasal prong electrodes of a nasal cannula uses the data from the flex sensors or the nasal prong electrodes to determine if the nasal cannula is properly positioned. Systems including the devices and a computer system are disclosed. Methods of using the devices and systems are disclosed.

### CROSS REFERENCE TO RELATED APPLICATIONS

This application is based upon and claims benefit of priority to U.S. Provisional Application No. 63/657,403, filed Jun. 7, 2024, which is hereby incorporated by reference herein in its entirety.

### FIELD

Disclosed herein is a device and system that identifies improper wear of a nasal cannula and alerts the user of such improper wear.

### BACKGROUND

Oxygen therapy involves the prescribed administration of oxygen and/or a flow rate for oxygen to be delivered for effective breathing via a nasal cannula having prongs resting in the nose, via a face mask, or via a tube placed in the trachea of a patient.

Home oxygen therapy has been shown to decrease mortality and improve quality of life for patients with Chronic Obstructive Pulmonary Diseases (COPD) and hypoxemia. Home oxygen therapy is prescribed to those who have been lifelong smokers, and some who are actively smoking. For home use, oxygen concentrators are used to pull oxygen from the surrounding air, never requiring a refill, but its use is dictated by a prescribed flow rate for each patient.

For portable use, oxygen is considered a medicine stored as a gas or liquid in special tanks. Lightweight concentrators are also available, eliminating the need for a special tank.

Because oxygen accelerates combustion and is a known fire hazard, COPD patients who smoke and use oxygen therapy in their homes or other enclosed spaces risk having a fire-related incident.

The prongs of a cannula are intended to direct oxygen into the nose. However, a significant amount of oxygen exits the nose and constantly leaks out and bathes the lower face. An oxygen-enriched environment facilitates ignition and combustion of any material. The buildup of oxygen in an enclosed space is often caused by non-compliant behavior, such as removing one's cannula without turning off the machine. Additionally, patients suffering from hypoxemia due to, for example, congestive heart failure and COPD are at risk when nasal cannula are not used properly. For example, a relatively large proportion of COPD patients using continuous supplemental oxygen suffer nasal cannula dislodgment during sleep on a weekly basis. Nasal cannula dislodgement predisposes COPD patients to exacerbations that may require emergency room treatment.

As shown in FIG. 1, known devices 20 for monitoring and/or controlling flow of gas from a gas source may include and rely on a flex sensor 22 used to determine that the nasal cannula was worn properly around the user's ear. As shown in FIG. 2, the flex sensor 22 may be coupled to a microcontroller-based device 24. In this example, the microcontroller-based device 24 is an Arduino Nano. The device 20 may further comprise at least one actuator 26 and power source 28 connected to the microcontroller-based device 24. In this example, the at least one actuator 26 may comprise servo motors (or “servos”) and the power source 28 comprises batteries. The device 20 may be configured to sense, via the flex sensor 22, that the nasal cannula is not properly worn around the car and may alert the user of improper wear of the nasal cannula by cutting off the flow of oxygen using the at least one actuator 26 and a cannula kinking technique based on the flex sensor 22. The lack of flow of oxygen would in turn cause the home oxygen concentrator to trigger and begin alarm sounds. The device 20 for controlling flow of gas from a gas source may comprise embodiments and details disclosed in GAS FLOW CONTROL DEVICE, PCT/US22/52526, which is incorporated fully herein by reference. Potential disadvantages of the device 20 may include having all floating components (except for the power switch), employing a single rigidly connected flex sensor, containing no calibration sequence for the flex sensors, and limited battery life. There is clearly an opportunity to increase the safety of oxygen therapy by reducing the potential for fire-related incidents as well as correcting dislodgement of nasal cannula and thereby mitigating concomitant complications.

### SUMMARY

An example device for determining when a nasal cannula is positioned properly on a user is disclosed. The device comprises a connecting tube configured to couple to a gas source. A first nasal prong is coupled to the connecting tube and configured to insert into a first nostril of a user. The first nasal prong comprises a first nasal prong electrode. A second nasal prong is coupled to the connecting tube and is configured to insert into a second nostril of the user. The second nasal prong comprises a second nasal prong electrode. A first resistor has a predetermined, known resistance value and is electrically coupled to the first nasal prong electrode and the second nasal prong electrode. The first nasal prong electrode and the second nasal prong electrode form a second resistor having a variable resistance value that changes depending on contact between the first and second nasal prong electrodes and skin within the first and second nostrils of the user. A voltage source is configured to introduce an input voltage across the first resistor and the second resistor. A voltage sensing component is configured to detect an output voltage between the first resistor and the second resistor.

An example device for monitoring flow of gas from a gas source to a patient is disclosed. The device comprises a housing. A microcontroller is positioned within the housing. The microcontroller is configured to communicate with at least one flex sensor and at least one nasal prong electrode. The microcontroller is configured to:
receive data from at least one of at least one flex sensor or at least one nasal prong electrode;
determine, based on the received data, whether the nasal cannula is not properly positioned; and
cause an alarm to be triggered when the microcontroller determines the nasal cannula is not properly positioned.

An example method for determining when a nasal cannula is positioned properly on a user is disclosed. The method comprises:
introducing an input voltage across a first resistor having a predetermined, known resistance value, a first nasal prong electrode of a nasal cannula configured to be positioned within a first nostril of the user, and a second nasal prong electrode of the nasal cannula configured to be positioned within a second nostril of the user, wherein the first nasal prong electrode and the second nasal prong electrode form a second resistor having a variable resistance value that changes depending on contact between the first and second nasal prong electrodes and skin within the first and second nostrils of the user; and
determining an output voltage between the first resistor and the second resistor.

### DESCRIPTION OF THE DRAWINGS

FIG. 1 shows an example known device.

FIG. 2 shows an example device according to the disclosure.

FIG. 2A is a block diagram of an example voltage divider incorporated into the device according to the disclosure.

FIG. 3 shows an example device according to the disclosure.

FIGS. 4A-5 show an example device according to the disclosure.

FIG. 6 shows an example charger.

FIG. 7 shows an example schematic for the example device.

FIG. 8 shows an example device according to the disclosure.

FIG. 9 shows an example battery connection.

FIG. 10 shows a diagram comparing approximate sizes of example devices with the size of a 9V battery.

FIG. 11A shows an example device according to the disclosure.

FIG. 11B shows an example printed circuit board (PCB) for the example device shown in FIG. 11A.

FIG. 12 shows an example charge port safety.

FIG. 13 shows an example system according to the disclosure.

FIG. 14 shows an example user interface.

FIG. 15 shows an example schematic for the example device.

FIG. 16 is a block diagram of an example control system comprising a computing device as disclosed herein for controlling the devices according to the disclosure.

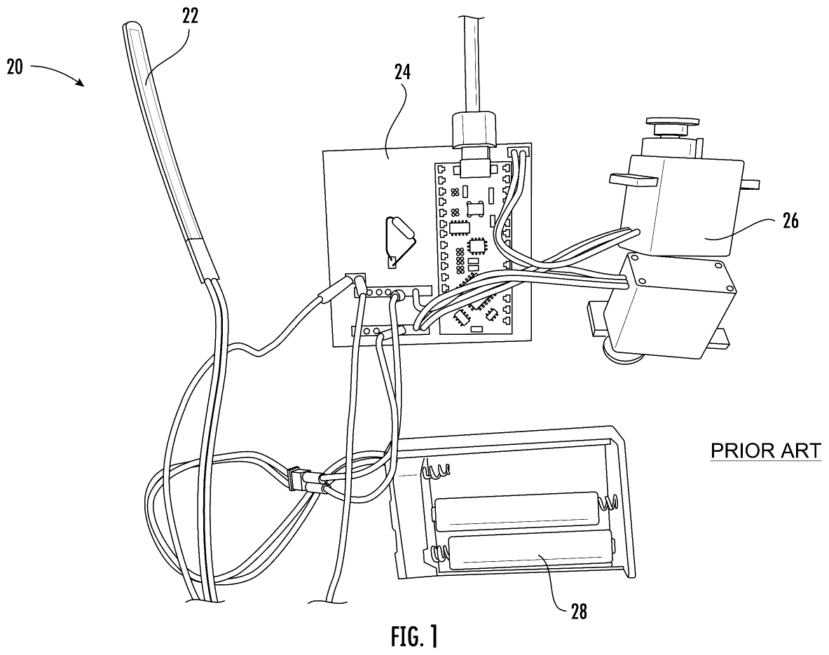

### DETAILED DESCRIPTION

The present invention now will be described more fully hereinafter with reference to the accompanying drawings, in which some, but not all embodiments of the invention are shown. Indeed, this invention may be embodied in many different forms and should not be construed as limited to the embodiments set forth herein; rather, these embodiments are provided so that this disclosure will satisfy applicable legal requirements. Like numbers refer to like elements throughout. It is to be understood that this invention is not limited to the particular methodology and protocols described, as such may vary. It is also to be understood that the terminology used herein is for the purpose of describing particular embodiments only, and is not intended to limit the scope of the present invention.

Many modifications and other embodiments of the invention set forth herein will come to mind to one skilled in the art to which the invention pertains having the benefit of the teachings presented in the foregoing description and the associated drawings. Therefore, it is to be understood that the invention is not to be limited to the specific embodiments disclosed and that modifications and other embodiments are intended to be included within the scope of the appended claims. Although specific terms are employed herein, they are used in a generic and descriptive sense only and not for purposes of limitation.

As used herein the singular forms “a,” “an,” and “the” include plural referents unless the context clearly dictates otherwise. For example, unless the context dictates otherwise, use of the term “a signal” can represent disclosure of embodiments in which only a single such signal is provided, as well as disclosure of embodiments in which a plurality of such signals are provided, and so forth.

All technical and scientific terms used herein have the same meaning as commonly understood to one of ordinary skill in the art to which this invention belongs unless clearly indicated otherwise.

Ranges can be expressed herein as from “about” one particular value, and/or to “about” another particular value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint. Optionally, in some aspects, when values are approximated by use of the antecedent “about,” it is contemplated that values within up to 15%, up to 10%, up to 5%, or up to 1% (above or below) of the particularly stated value can be included within the scope of those aspects. Similarly, in some optional aspects, when values are approximated by use of the terms “approximately,” “substantially,” or “generally,” it is contemplated that values within up to 15%, up to 10%, up to 5%, or up to 1% (above or below) of the particular value can be included within the scope of those aspects. When used with respect to an identified property or circumstance, “substantially” or “generally” can refer to a degree of deviation that is sufficiently small so as to not measurably detract from the identified property or circumstance, and the exact degree of deviation allowable may in some cases depend on the specific context.

As used herein, the terms “optional” or “optionally” mean that the subsequently described event or circumstance may or may not occur, and that the description includes instances where the event or circumstance occurs and instances where it does not.

The following description supplies specific details in order to provide a thorough understanding. Nevertheless, the skilled artisan would understand that the system and associated methods of using the system can be implemented and used without employing these specific details. Indeed, the system and associated methods can be placed into practice by modifying the illustrated apparatus and associated methods and can be used in conjunction with any other apparatus and techniques conventionally used in the industry.

### INTRODUCTION

Described herein are example devices 30, 10, 100 and system 1000 configured to identify improper wear of a nasal cannula and alert the user to ensure proper delivery of a gas, optionally oxygen, from a gas source, optionally a home oxygen concentrator. The example devices 30, 10, 100 and system 1000 disclosed are expected to improve detection of the placement of the nasal prongs to confirm the nasal prongs are properly positioned within the nostrils of the user.

The skin inside the nostrils has low electrical resistance because it is thin, moist, and rich in blood vessels. Unlike the outer layers of skin, which are typically dry and thick, the mucous membrane lining the nostrils does not provide much insulation against electrical current. Moisture, including natural mucus, significantly reduces resistance, allowing electrical current to pass through more easily thereby making the nasal cavity more conductive than dry skin on the surface of the body. The electrical resistance inside of the nostrils may be about 100 k ohms. The resistance of the skin inside the nostrils may be used to determine if the nasal prongs of the nasal cannula are properly positioned within the nostrils.

### Devices

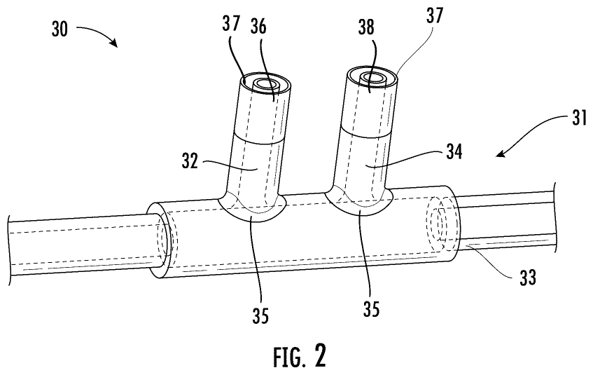

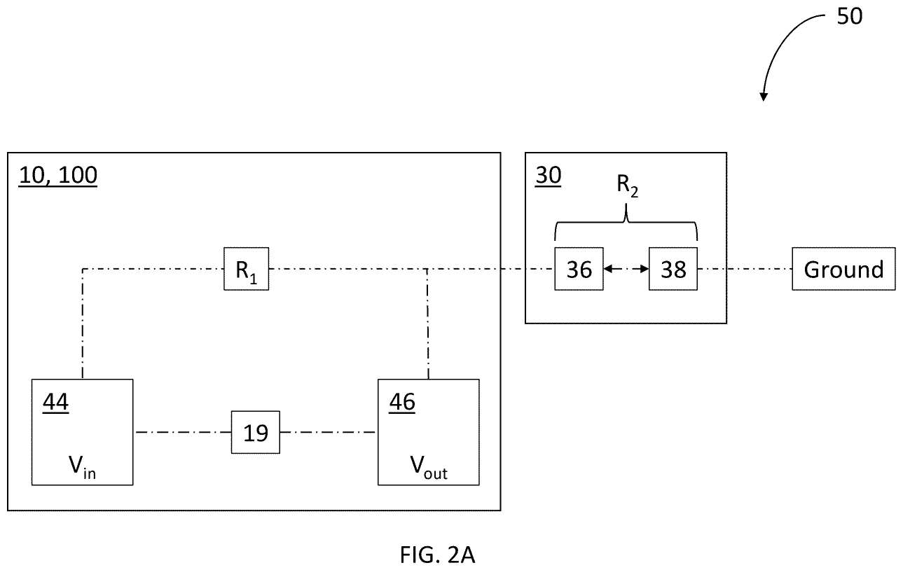

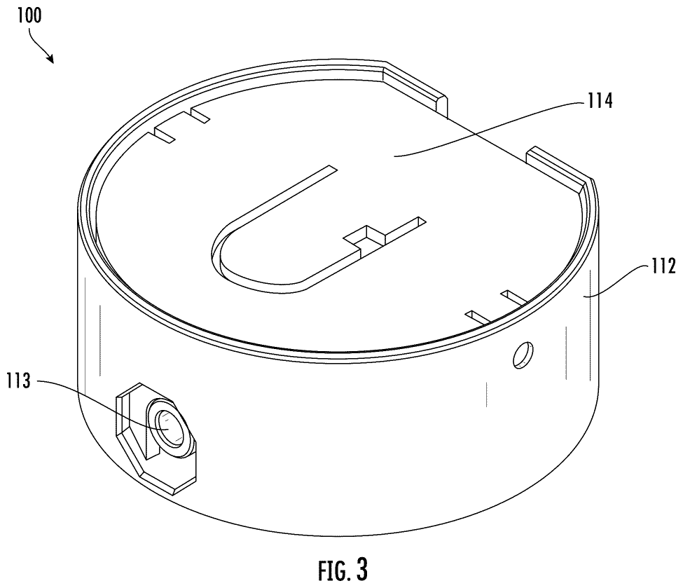

FIG. 2 shows an example device 30, such a nasal cannula, for monitoring flow of gas, optionally oxygen, from a gas source. The device 30 may comprise a nasal cannula 31. The device 30, optionally the nasal cannula 31, may comprise at least one nasal prong electrode 36, 38. Optionally, standard alloys, for example copper, steel, etc., may be used for the electrodes 36, 38 to make them safe for skin contact. Each nasal prong electrode 36, 38 may be configured to apply a voltage and/or detect current flow. As shown in FIG. 3, the device 30 may comprise at least one nasal prong 32, 34 configured to insert into a user's nostrils. The nasal cannula 31 may comprise a connecting tube 33. Each nasal prong 32, 34 may be coupled to the connecting tube 33. Each nasal prong 32, 34 may comprise a proximal end 35 configured to couple to the connecting tube 33 and a distal free end 37 configured to insert into a nostril. A nasal prong electrode 36, 38 may be positioned adjacent (optionally immediately adjacent) to each distal free end 37 of the nasal prong 32, 34. Each nasal prong 32, 34 may comprise at least one nasal prong electrode 36 or 38. In this example, the device 30 comprises a first nasal prong 32 and a second nasal prong 34. The first nasal prong 32 may be configured to insert into a first nostril of the user and the second nasal prong 34 may be configured to insert into a second nostril of the user. The first nasal prong 34 may comprise a first nasal prong electrode 36. The second nasal prong 34 may comprise a second nasal prong electrode 38. The device 30 may be configured to ensure the nasal prongs 32, 34 are properly positioned in the user's nose via the nasal prong electrodes 36, 38. Each nasal prong electrode 36, 38 may apply a voltage and/or detect a current flow. A resistance may be determined and/or calculated based on the applied voltage and detected current flow to determine if the nasal prong electrode 36, 38 are contacting skin inside the nostrils thereby ensuring the nasal prongs 32, 34, which include the nasal prong electrodes 36, 38, are positioned within the nostrils.

As shown in FIG. 2A, the first nasal prong electrode 36 and the second nasal prong electrode 38 of the device 30 may be incorporated into a voltage divider 50 which uses the skin resistance on the nasal prongs 32, 34 as an element in the divider 50 thereby acting as an indicator of proper wear of the nasal cannula 31. In one example, a known first resistor R₁ may be connected to the nasal prong electrodes 36, 38 across a voltage source. Optionally, the resistor R₁ may be a standard resistor known in the art. Optionally, the first resistor R₁ has a predetermined, known value. The value may be from about 1 M ohm to about 10 M ohm. The first resistor R₁ may be electrically coupled to the nasal prong electrodes 36, 38. The nasal prong electrodes 36, 38 may form a second resistor R₂ having a variable resistance value (skin). A safe voltage Vᵢₙ (optionally, for example, less than 5 V) may be introduced and a voltage out Vₒᵤₜ may be measured between the known first resistor R₁ and the variable second resistor R₂. If the nasal prong electrodes 36, 38 are in proper contact with the low-resistance skin inside the nostrils, the voltage out Vₒᵤₜ may change in a predictable way. If the nasal prong electrodes 36, 38 are not properly contacting the skin inside the nostrils, the voltage out Vₒᵤₜ may signal that the electrodes 36, 38 are not in proper contact with skin within the user's nostrils, and ultimately, that the nasal cannula 31 is not being worn correctly. This configuration creates a voltage divider with a known resistor R₁ and a variable resistor R₂ create by the nasal prong electrodes 36, 38 along with the user's skin between the electrodes 36, 38 as the unknown element. Using the output voltage Vₒᵤₜ, the position of the nasal cannula 31 (for example, whether the nasal prongs are positioned within the nostrils of a user) may be determined because contact with skin inside the nostrils may lower resistance, changing the voltage in a detectable way.

FIGS. 3-12 show an example device 10, 100 according to the disclosure for monitoring flow of gas from a gas source. The device 10, 100 may be configured to communicate with the at least one flex sensor 22, which may be the flex sensor described in and configured to operate as described in GAS FLOW CONTROL DEVICE, PCT/US22/52526, and/or the device 30 including the at least one nasal prong electrode 36, 38. Advantageously, the device 10, 100 may be used with the flex sensor 22 and/or the device 30 described herein. The device 10, 100 may comprise an alarm system that is activated based on data from the at least one flex sensor 22 and/or the at least one nasal prong electrodes 36, 38 to alert the user when the nasal cannula is not properly positioned.

As shown in FIG. 2A, the device 10, 100 may utilize the device 30, optionally the nasal cannula 31, to determine when the device 30 is positioned properly on a user (for example, when the nasal prong electrodes 36, 38 are positioned within the nostrils of the user and in contact with skin inside the nostrils). The device 10, 100 may incorporate the voltage divider 10 to determine when the device 30 is positioned properly on the user. The device 10, 100 may comprise the known resistor R₁. The device 10, 100 may comprise a voltage source 44 configured to introduce the input voltage Vᵢₙ across the first resistor R₁ and/or the second resistor R₂ (i.e. the nasal prong electrodes 36, 38). The voltage source 44 may be a known voltage source known in the industry. Optionally, the voltage source 44 may comprise at least one of a microcontroller GPIO pin, battery, regulator, and/or digital-to-analog converter. The device 10, 100 may comprise a voltage sensing component 46 configured to detect the output voltage Vₒᵤₜ. The voltage sensing component 46 may be a known voltage sensing component known in the industry. Optionally, the voltage sensing component 46 may be at least one of an analog-to-digital converter, amplifier, voltmeter, and/or multimeter.

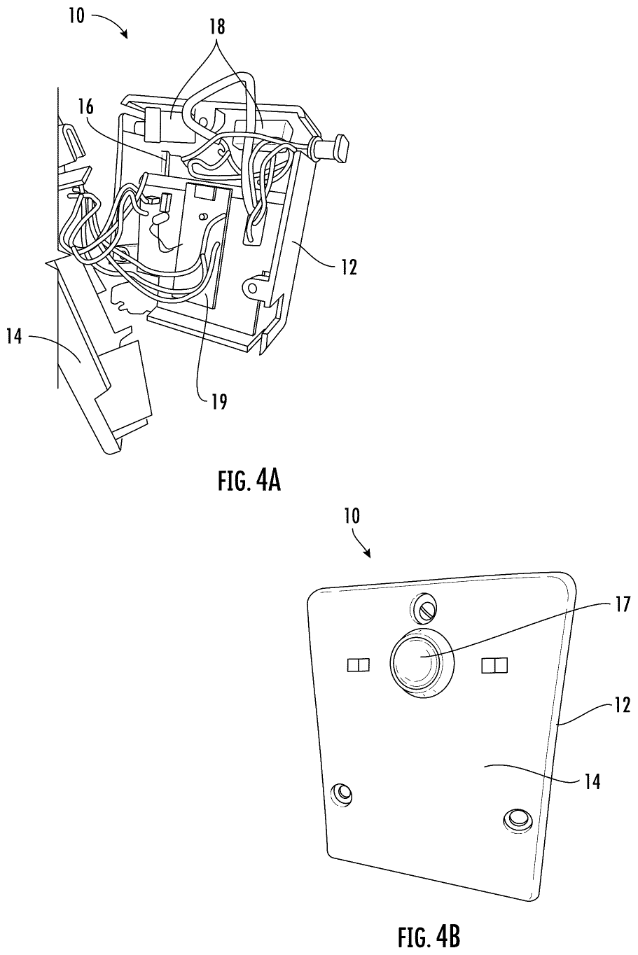

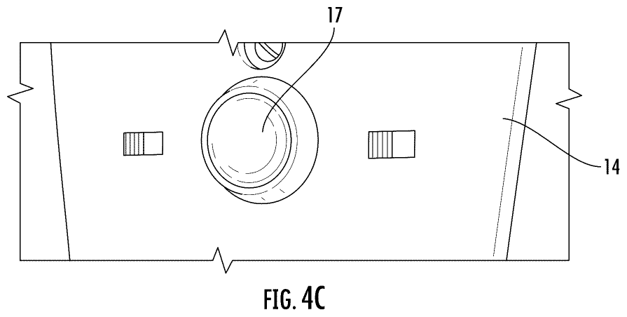

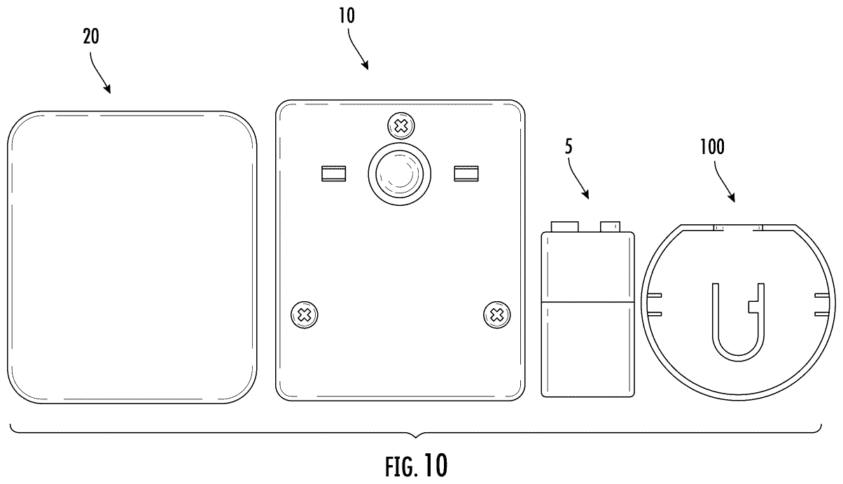

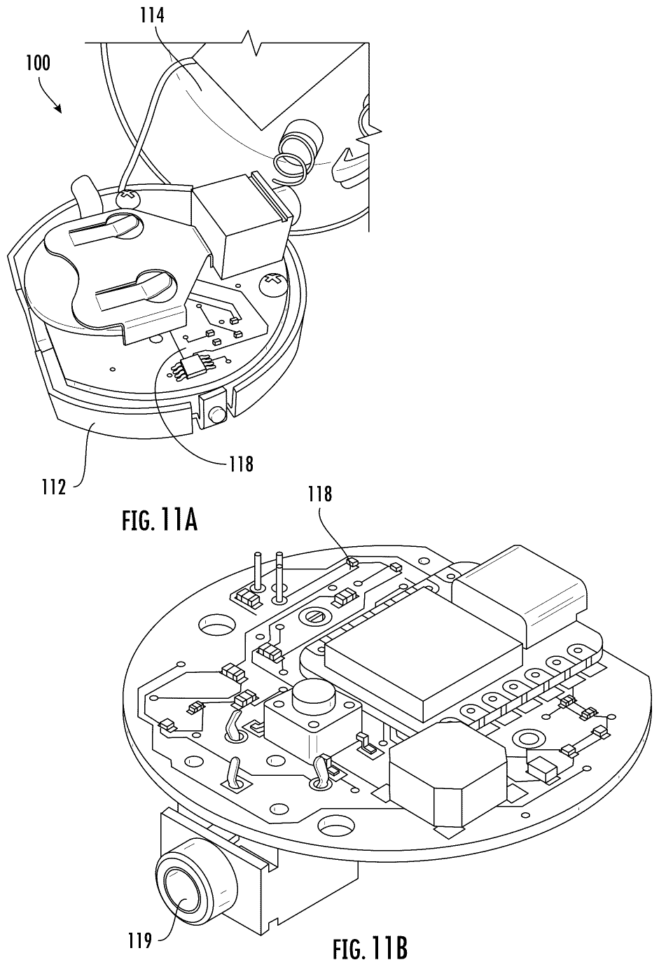

As shown in FIGS. 3, 4A, and 11A, the device 10, 100 may comprise a housing 12, 112. Optionally, as shown in FIGS. 3-4B and 11A, the device 10 may comprise a lid 14, 114 removably coupled to the housing 12, 112. FIGS. 3 and 4B show the lid 14, 114 coupled to the housing 12, 112 to contain and protect the components within the housing 12, 112. FIGS. 4A and 11A show the lid 14, 114 not coupled to the housing 12 to show the interior of the device 10, 100. Although it may not be shown in the figures, it is understood device 100 may comprise any of the components of device 10 and device 10 may comprise any of the components of device 100.

As shown in FIG. 4A, the device 10 may comprise a protoboard 16, optionally coupled or fastened to the housing 12, that may be used to rigidly mount other components including at least one actuator 18, microcontroller-based device 19, and/or power source (not visible in FIG. 4A or 4B). The at least one actuator 18 may comprise servos. The at least one actuator 18 may be configured to activate or trigger the alarm system and/or prevent that gas from flowing from the gas source to the nasal prongs 32, 34. The power source may be a lithium-ion battery. The power source may passively introduce the input voltage Vᵢₙ. The power source may deliver about 5V to the device 10, and the power source capacity may be about 3000 mAh. The device may comprise a Micro USB charge port to charge the power source.

As shown in FIGS. 4B and 4C, the device 10 may comprise a switch 17. Optionally the switch 17 may comprise a button. The switch 17 may allow the device 10 to create an offset reference value to calibrate the sensors. For example, a user may properly position the nasal cannula and activate the switch 17 to measure, determine, and store the curvature of the nasal cannula to “calibrate” the flex sensors 22. Optionally, the switch may allow the device 10 to determine a voltage out Vₒᵤₜ when the nasal prongs are properly positioned within the nostrils of the user to set a baseline or threshold to use to determine that the nasal prongs are not properly positioned within the nostrils when a real time determination of the voltage out Vₒᵤₜ shifts.

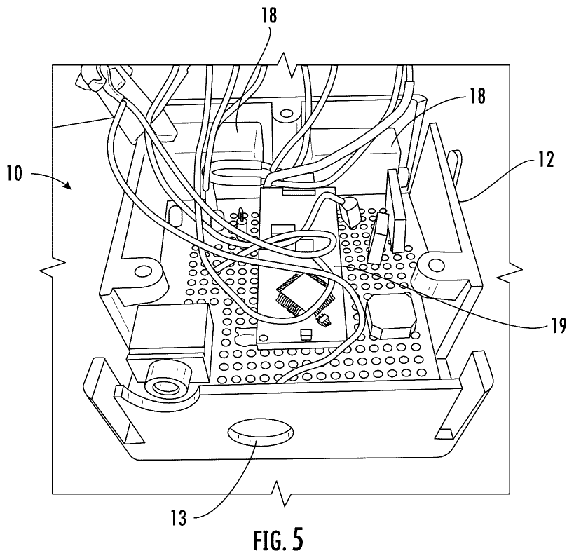

As shown in FIGS. 3 and 5, the device 10, 100 may comprise a headphone jack 13, 113. The device 10 may utilize a headphone jack 13, 113 where sensors or electrodes may be configured offline and then plugged in. Dual flex sensors 22 or the nasal prong electrodes 36, 38 may be accepted via the headphone jack 13, 113. Advantageously, different sensors may be used with the device 10. A mode switch may be used to switch between the flex sensors or the nasal prong electrode sensors.

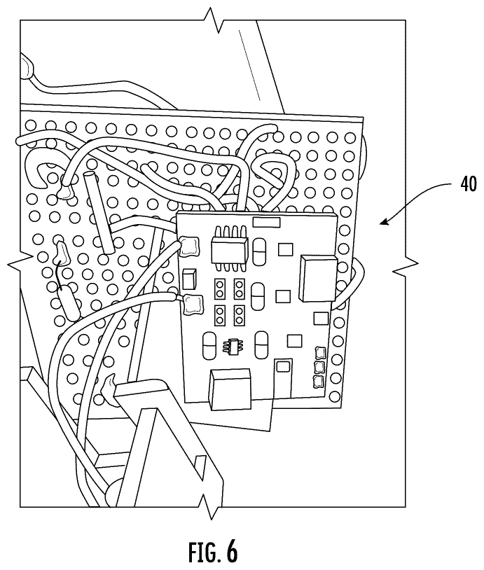

As shown in FIG. 6, the device 10 may comprise a charging module 40. Optionally, the charging module 40 is a J5019 charger. The charging module 40 may be configured to charge the power source of the device 10. Optionally, the charging module 40 may be configured to charge a lithium-ion battery. Optionally, the charging module 40 may deliver about 5V.

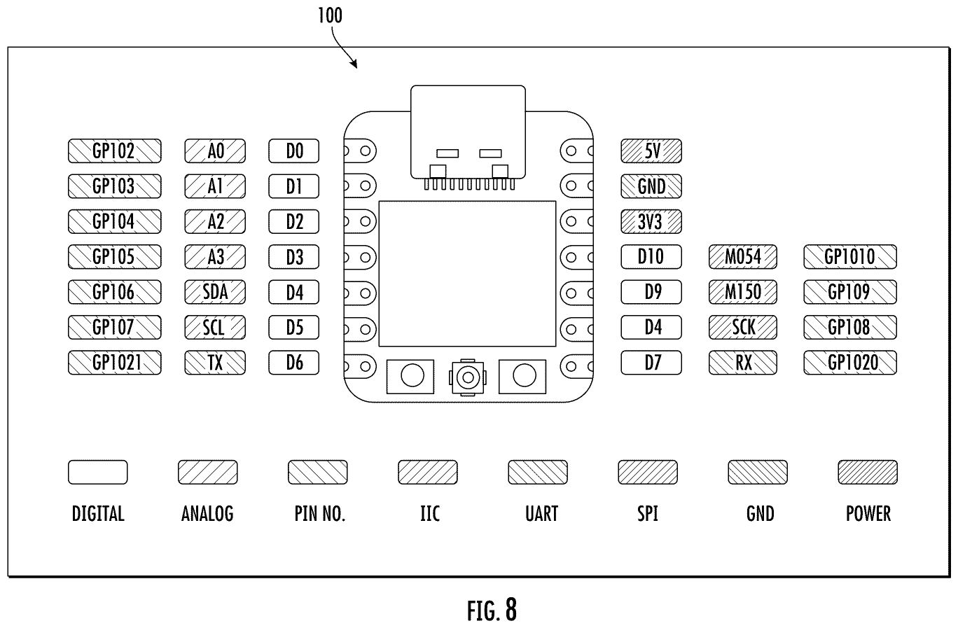

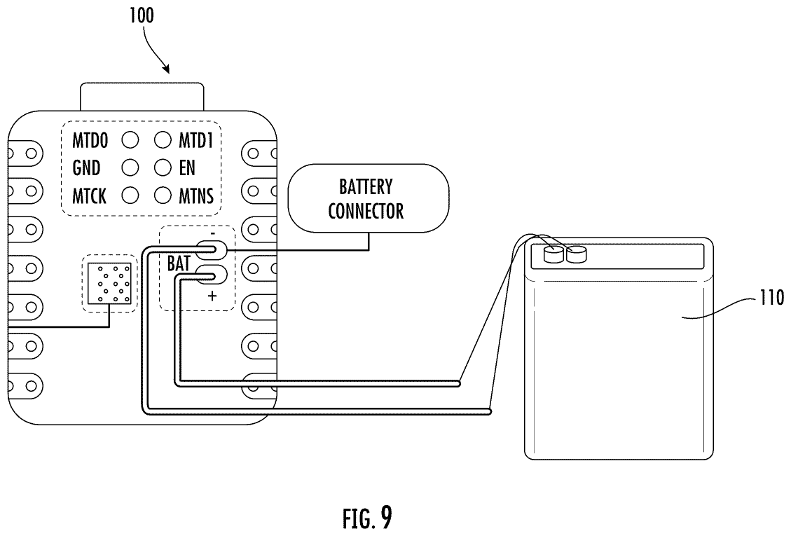

FIG. 8 shows another configuration of the example device 100 according to the disclosure. The device 100 may comprise a microcontroller-based device based on the ESP32C3. The microcontroller based device may comprise wireless capabilities as well as extremely low power draw. Optionally, the microcontroller based device may be a Seeduino XIAO-ESP32-C3. The microcontroller based device may comprise a USB port and battery charging integrated circuit. As shown in FIG. 9, the device 100 may comprise a battery connection 110. Optionally, the battery connection may be configured for a Seeduino XIAO-ESP32-C3.

Optionally, the device 100 may comprise the following features: integrated charging, single board design with no external wires, single button functionality, Wi-Fi and Bluetooth capabilities, text message notification capabilities through Twilio, support for nasal prong electrodes and flex sensors through single port, automatic detection of which sensor is being used, captive portal-based web user interface through long press of button, configurable threshold and sensitivity values for sensors and/or electrodes, real time sensor readings through captive portal webpage, configurable alarm frequencies, weigh 32 grams, low battery detection, 1 week battery life, transient voltage protection on input, and mounted coin cell battery.

FIG. 10 shows the approximate size of different devices 20, 10, 100 for monitoring flow of gas from a gas source as compared to a 9V battery 5.

FIG. 11A shows an example device 100 according to the disclosure. The device 100 may comprise a housing 112. Optionally, a lid 114 may removably couple with the housing 112. FIG. 11A shows the lid 114 removed from the housing 112 to show the interior of the device. The device 100 may comprise a printed circuit board (PCB) 118 positioned within the housing 112. As shown in FIG. 11B, the PCB 118 may be coupled to a headphone jack 113. Optionally, the PCB may be a 4-layer board. Populating and reflowing the board may be done relatively quickly, for example in an hour or even much more quickly.

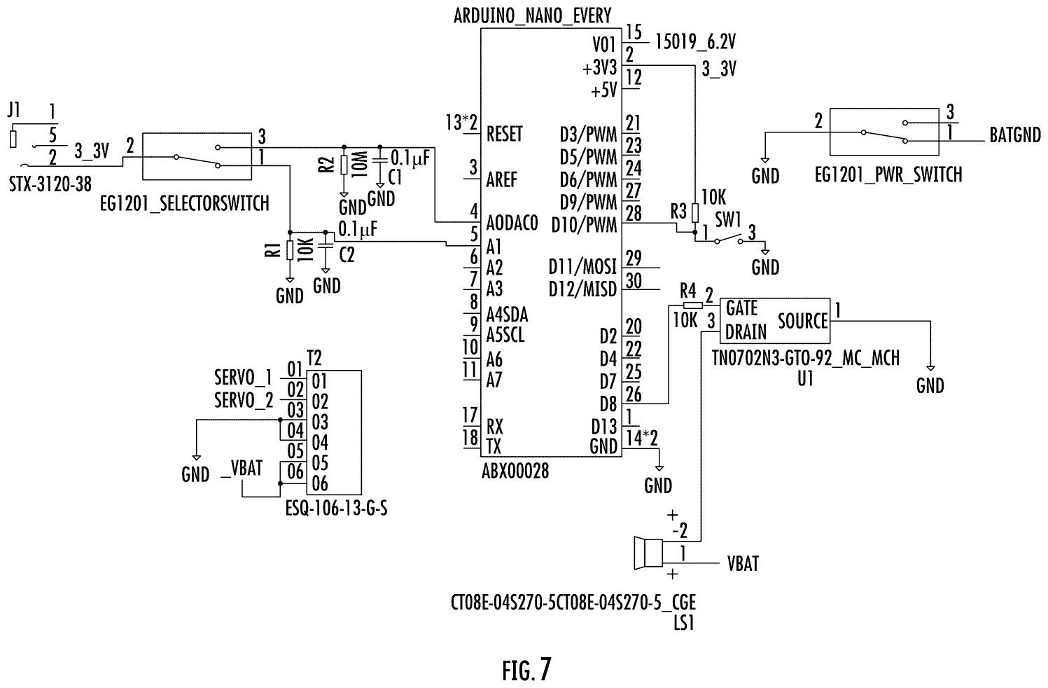

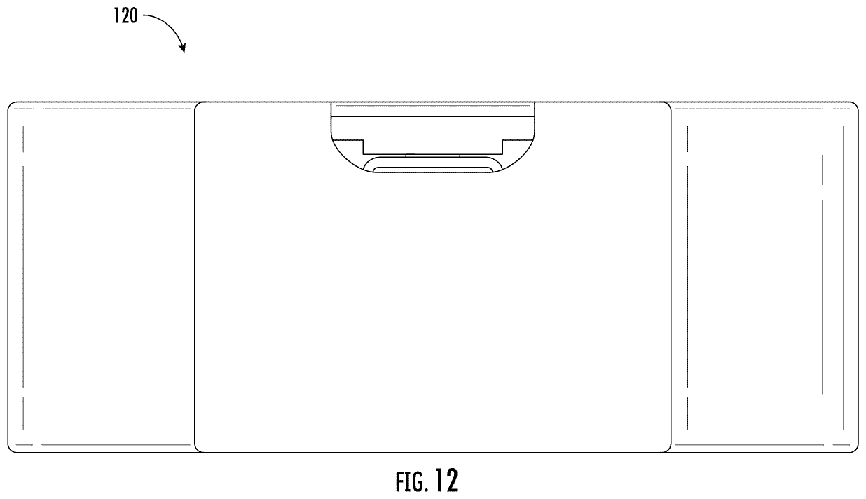

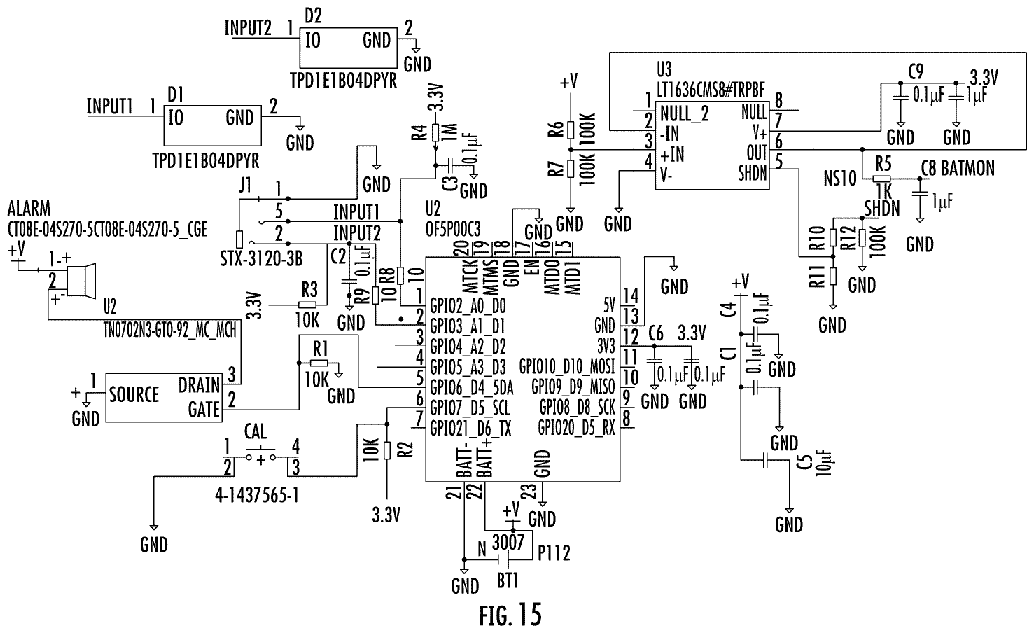

FIG. 12 shows an example charge port safety 120. The charge port safety 120 may comprise a spring hinged design that requires the sensor, for example the nasal prong electrode sensor, to be unplugged to access the charging port. This feature may ensure the device 100 is charged safely as an AC voltage could bypass all electrical safety measures if the nasal prong electrode sensors are used while the device 100 is charging. FIGS. 7 and 15 show example schematics of the device 10, 100.

The microcontroller based device (for example device 19) of the device 10, 100 may communicate with and/or comprise the voltage source 44 and/or the voltage sensing component 46. The microcontroller based device 19, voltage source 44, and/or the voltage sensing component 46 may form a real-time loop such that the microcontroller continuously processes data from the voltage source 44 and the voltage sensing component 46. The microcontroller based device 19 of the device 10, 100 may comprise the resistor R₁. The microcontroller 19 may be configured to communicate with at least one flex sensor 22 and at least one nasal prong electrode 36, 38. The microcontroller 19 may be configured to receive data from at least one of at least one flex sensor 22 and/or at least one nasal prong electrode 36, 38. The microcontroller may be configured to determine, based on the received data, whether the nasal cannula 31 is not properly positioned. The microcontroller 19 may be configured to cause the alarm to be triggered when the microcontroller determines the nasal cannula 31 is not properly positioned. The microcontroller may be configured to determine, based on the received data of at least one flex sensor 22, a flex level. The microcontroller may be configured to determine whether the nasal cannula 31 is properly positioned based on the flex level. The microcontroller may determine the flex level and whether the nasal cannula is properly positioned based on the flex level as described in GAS FLOW CONTROL DEVICE, PCT/US22/52526. The microcontroller is configured to determine, based on the received data of the at least one nasal prong electrode 36, 38, at least one of a skin resistance level or a voltage output Vₒᵤₜ. The microcontroller may determine whether the nasal cannula 31 is properly positioned based on at least the skin resistance level or the voltage output. The microcontroller may be configured to introduce the voltage input Vᵢₙ across the resistor R₁ and the at least one nasal prong electrode 36, 38. The voltage output Vₒᵤₜ determined by the microcontroller may be based on the voltage input Vᵢₙ, the resistor R₁, and the at least one nasal prong electrode 36, 38. The microcontroller of the device 10, 100 may be communicatively coupled to the actuator of the device 10, 100. The actuator of the device 10, 100 may be configured to prevent the flow of the gas from the gas source to the patient and/or activate the alarm when the microcontroller determines the nasal cannula 31 is not properly positioned.

### System

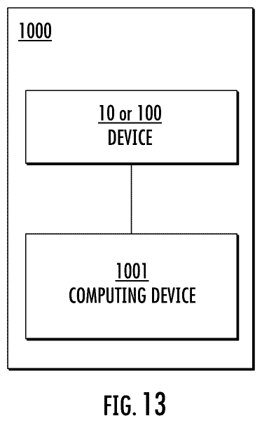

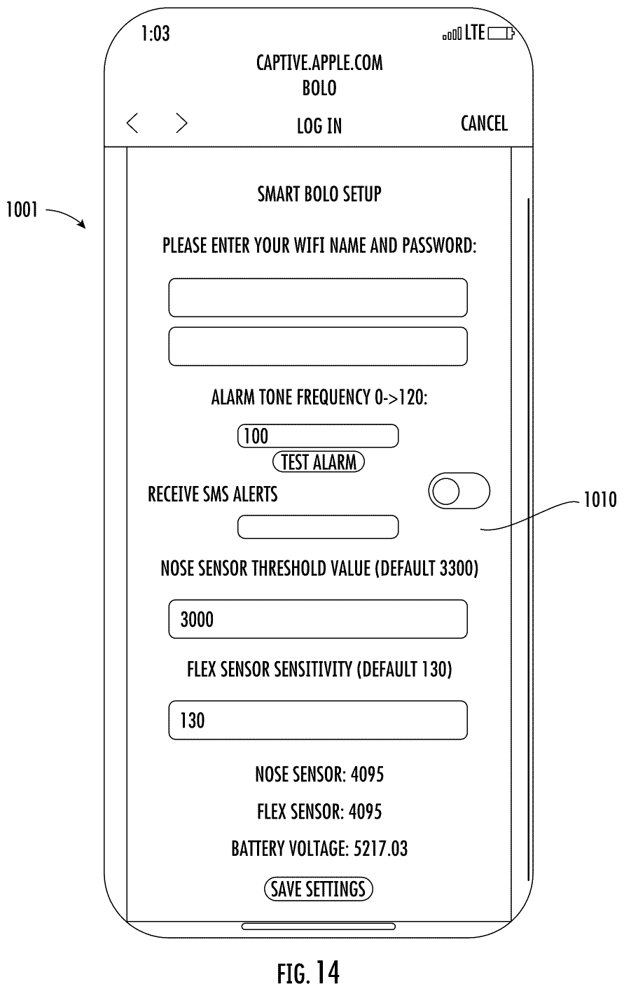

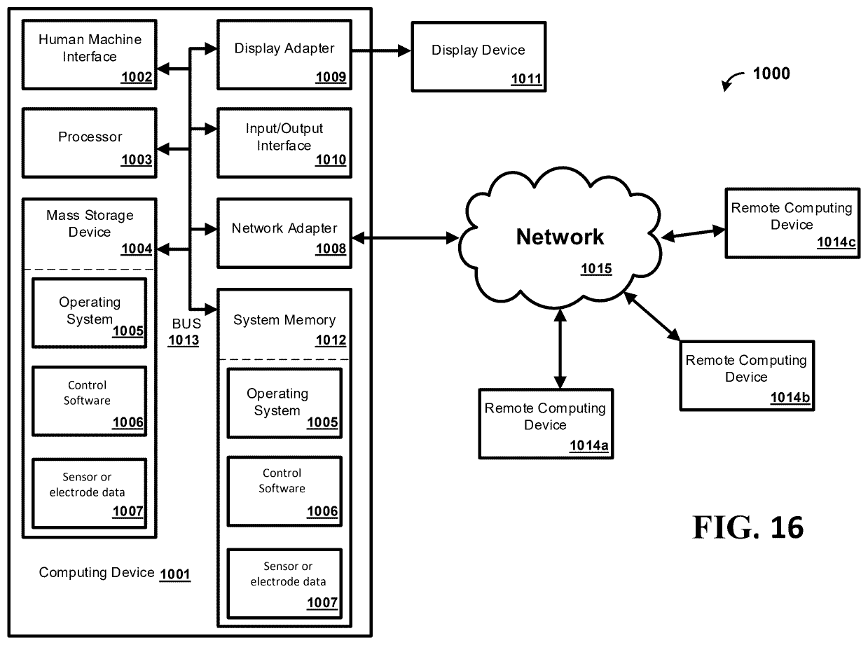

FIGS. 13 and 16 show an example control system 1000 that identifies improper wear of a nasal cannula and alerts the user. The control system 1000 comprises a computing device 1001 in communication with a device 10, 100. Optionally, the computing device 1001 may be positioned within the housing 12, 112 of the device 10, 100. Optionally, the computing device 1001 may be remote to the device 10, 100. The computing device 1001 may send and/or receive data to and/or from the device 10 or 100. For example, the computing device 1001 may receive data corresponding to the data received from the nasal prong electrodes 36, 38 and/or the dual flex sensors 22. Optionally, the computing device 1001 may send data corresponding to a threshold value or range (for example, a threshold voltage out Vₒᵤₜ value or range) inputted by a user to the device 10 or 100. Optionally, the computing device 10 or 100 is a smart phone, laptop, or desktop computer. Optionally, a user may activate the switch of the device, for example, may hold down the button, to cause the device 10 or 100 to generate a WiFi hotspot that the computing device 1001 may connect to. The computing device 1001 may store software comprising an accompanying phone or tablet application (app) which may serve as a monitoring tool for the user of the device 10 or 100 or serve as a programing tool for the user of the device 10 or 100 to change settings of the device 10 or 100. The app may be compatible with both iOS and Android devices, ensuring accessibility for a broad range of users. FIG. 14 shows an example user interface 1010. The user interface 1010 may be reprogrammed as experimentation and testing is done. If a user does not set up the device 10 or 100, the computing device 1001 and/or the device 10 or 100 may use default values for parameters such as nasal prong electrode threshold, voltage out threshold, flex sensor threshold, alarm frequency, and/or SMS Alerts Off.

The computing device 1001 may be configured to determine if the nasal prongs of a nasal cannula are properly positioned within the nostrils of a user. If the computing device 1001 determines the nasal prongs are not properly positioned within the nostrils of the user, the computing device 1001 may cause the alarm system of the device 10, 100 to alarm the user, optionally, via a sound, a visual indicator, and/or vibration. If the computing device 1001 determines the nasal prongs are not properly positioned within the nostrils of the user, the computing device may be configured to cause the device 10, 100, via the actuators, to disrupt the flow of a gas from a gas source. The actuators may be known actuators in the industry. Optionally, the actuator is a servos configured to disrupt the flow of the gas from the gas source via known cannula kinking techniques. The computing device 1001 may be configured to determine if the nasal prongs of a nasal cannula are properly positioned within the nostrils of a user via data from the flex sensors 22 and/or the at least one nasal prong electrode 36, 38. Optionally, the computing device 1001 may be configured to compare the data from the flex sensors 22 and/or the at least one nasal prong electrode 36, 38 to a threshold value or range to determine if the nasal prongs of a nasal cannula are properly positioned within the nostrils of a user. The computing device 1001 may be configured to cause the device 10, 100 to provide voltage to the at least one nasal prong electrode 36, 38. The computing device 1001 may be configured to determine a resistance based on data from the nasal prong electrodes 36, 38. The computing device 1001 may be configured to determine a voltage out Vₒᵤₜ between a known resistor R₁ and variable resistor R₂ (the nasal prong electrodes 36, 38 and skin between). The computing device 100 may cause the voltage source 44 to introduce the input voltage Vᵢₙ. The computing device 100 may compare the output voltage Vₒᵤₜ to a threshold value to determine whether the first nasal prong 32 is positioned within the first nostril of the user and the second nasal prong 34 is positioned within the second nostril of the user. The threshold value may be a range (for example, the threshold range may comprise a maximum threshold and a minimum threshold. The threshold value may be a maximum value. The threshold value may be a minimum value. Whether the threshold value is a maximum value or a minimum value may depend on the configuration of the first resistor R₁ with respect to the second resistor R₂ the first nasal electrode 36 and second nasal electrode 38. The threshold values may be determined and inputted by a user (optionally, via software). Optionally, the threshold value may be determined based on a determined (optionally, measured) voltage out Vₒᵤₜ when the first nasal prong is properly positioned within the first nostril of the user and the second nasal prong is properly positioned within the second nostril of the user. The threshold value may be a percentage of the determined voltage out Vₒᵤₜ when the first nasal prong is properly positioned within the first nostril of the user and the second nasal prong is properly positioned within the second nostril of the user.

The computing device 1001 may be configured to cause the microcontroller 19 of the device 10, 100 to introduce the voltage input Vᵢₙ across the resistor R₁ and/or the at least one nasal prong electrode 36, 38. The computing device 1001 may be configured to receive data corresponding to the output voltage Vₒᵤₜ and compare the output voltage Vₒᵤₜ to a threshold value (or range) to determine whether the first nasal prong 32 is positioned within the first nostril of the user and the second nasal prong 34 is positioned within the second nostril of the user. The computing device 1001 may be configured to cause the microcontroller of the device 10, 100 to trigger the alarm based on the comparison of the output voltage Vₒᵤₜ to the threshold value. The computing device 1001 may be configured to cause the actuators of the device 10, 100 to disrupt the flow of gas from the gas source.

FIG. 11 shows an example control system 500 including an exemplary configuration of the computing device 1001 for use with the devices 10, 100 disclosed herein. The computing device 1001 may comprise one or more processors 1003, a system memory 1012, and a bus 1013 that couples various components of the computing device 1001 including the one or more processors 1003 to the system memory 1012. In the case of multiple processors 1003, the computing device 1001 may utilize parallel computing. The bus 1013 may comprise one or more of several possible types of bus structures, such as a memory bus, memory controller, a peripheral bus, an accelerated graphics port, and a processor or local bus using any of a variety of bus architectures.

The computing device 1001 may operate on and/or comprise a variety of computer readable media (e.g., non-transitory). Computer readable media may be any available media that is accessible by the computing device 1001 and comprises, non-transitory, volatile and/or non-volatile media, removable and non-removable media. The system memory 1012 has computer readable media in the form of volatile memory, such as random access memory (RAM), and/or non-volatile memory, such as read only memory (ROM). The system memory 1012 may store data such as sensor and/or electrode data 1007 and/or program modules such as operating system 1005, and software 1006.

The computing device 1001 may also comprise other removable/non-removable, volatile/non-volatile computer storage media. The mass storage device 1004 may provide non-volatile storage of computer code, computer readable instructions, data structures, program modules, and other data for the computing device 1001. The mass storage device 1004 may be a hard disk, a removable magnetic disk, a removable optical disk, magnetic cassettes or other magnetic storage devices, flash memory cards, CD-ROM, digital versatile disks (DVD) or other optical storage, random access memories (RAM), read only memories (ROM), electrically erasable programmable read-only memory (EEPROM), and the like. Any number of program modules may be stored on the mass storage device 1004. An operating system 1005 and software 1006 may be stored on the mass storage device 1004.

A user may enter commands and information into the computing device 1001 using an input device. Such input devices comprise, but are not limited to, a joystick, a touchscreen display, a keyboard, a pointing device (e.g., a computer mouse, remote control), a microphone, a scanner, tactile input devices such as gloves, and other body coverings, motion sensor, speech recognition, and the like. These and other input devices may be connected to the one or more processors 1003 using a human machine interface 1002 that is coupled to the bus 1013, but may be connected by other interface and bus structures, such as a parallel port, game port, an IEEE 1394 Port (also known as a Firewire port), a serial port, network adapter 1008, and/or a universal serial bus (USB).

A display device 1011 may also be connected to the bus 1013 using an interface, such as a display adapter 1009. It is contemplated that the computing device 1001 may have more than one display adapter 1009 and the computing device 1001 may have more than one display device 1011. A display device 1011 may be a monitor, an LCD (Liquid Crystal Display), light emitting diode (LED) display, television, smart lens, smart glass, and/or a projector. In addition to the display device 1011, other output peripheral devices may comprise components such as speakers (not shown) and a printer (not shown) which may be connected to the computing device 1001 using Input/Output Interface 1010. Any step and/or result of the methods may be output (or caused to be output) in any form to an output device. Such output may be any form of visual representation, including, but not limited to, textual, graphical, animation, audio, tactile, and the like. The display 1011 and computing device 1001 may be part of one device, or separate devices.

The computing device 1001 may operate in a networked environment using logical connections to one or more remote computing devices 1014a,b,c. A remote computing device 1014a,b,c may be a personal computer, computing station (e.g., workstation), portable computer (e.g., laptop, mobile phone, tablet device), smart device (e.g., smartphone, smart watch, activity tracker, smart apparel, smart accessory), security and/or monitoring device, a server, a router, a network computer, a peer device, edge device or other common network node, and so on. The remote computing devices 1014a,b,c, can perform respective operations of the system. Logical connections between the computing device 1001 and a remote computing device 1014a,b,c may be made using a network 1015, such as a local area network (LAN) and/or a general wide area network (WAN), or a Cloud-based network. Such network connections may be through a network adapter 1008. A network adapter 1008 may be implemented in both wired and wireless environments. Such networking environments are conventional and commonplace in dwellings, offices, enterprise-wide computer networks, intranets, and the Internet. It is contemplated that the remote computing devices 1014a,b,c can optionally have some or all of the components disclosed as being part of computing device 1001. In various further aspects, it is contemplated that some or all aspects of data processing described herein can be performed via cloud computing on one or more servers or other remote computing devices. Accordingly, at least a portion of the system 1000 can be configured with internet connectivity.

### Operation of the Devices and Systems

A method of determining when a nasal cannula is positioned properly on a user (for example, the first nasal prong is position in the first nostril of the user and the second nasal prong is positioned in the second nostril of the user) may comprise using the device 10, 100 as described herein. The method may comprise:
introducing an input voltage across a first resistor having a predetermined, known resistance value, a first nasal prong electrode of a nasal cannula configured to be positioned within a first nostril of the user, and a second nasal prong electrode of the nasal cannula configured to be positioned within a second nostril of the user, wherein the first nasal prong electrode and the second nasal prong electrode form a second resistor having a variable resistance value that changes depending on contact between the first and second nasal prong electrodes and skin within the first and second nostrils of the user; and
determining an output voltage between the first resistor and the second resistor.

The method may comprise:
determining the output voltage when the first nasal prong electrode is positioned within the first nostril of the user and the second nasal prong electrode is positioned within the second nostril of the user.

The method may also comprise:
comparing the output voltage to the determine output voltage when the first nasal prong electrode is positioned within the first nostril of the user and the second nasal prong electrode is positioned within the second nostril of the user.

The method may also comprise:
comparing the output voltage to a threshold value; and/or
activating an alarm and/or preventing the flow of gas from a gas source to the first nasal prong and the second nasal prong when:
   the output voltage is greater than a maximum threshold value,
   the output voltage is less than a minimum threshold value, or
   the output voltage is offset at least a predetermined value from the output voltage is positioned within a first nostril of a user and the second nasal prong electrode is positioned within a second nostril of the user.

A number of advantages are expected to be achieved by the devices, systems, and methods described and claimed herein, including: lower oxygen (and other medical gas) use and waste; prevention of oxygen level build up without an alarm; allow for corrective measures when nasal cannula are dislodged during sleep; reduced fire risk; increased patient safety; increased safety for family, caregivers, neighbors, and pets. The devices described and claimed herein can be configured to work with existing home oxygen concentrators

All of the embodiments of the claimed invention described herein are provided expressly by way of example only. Innumerable variations and modifications may be made to the example embodiments described herein without departing from the concept of this disclosure. Additionally, the scope of this disclosure is intended to encompass any and all modifications and combinations of all elements, features, and aspects described in the specification and claims, and shown in the drawings. Any and all such modifications and combinations are intended to be within the scope of this disclosure.

### Exemplary Aspects

In view of the described products, systems, and methods and variations thereof, herein below are described certain more particularly described aspects of the invention. These particularly recited aspects should not however be interpreted to have any limiting effect on any different claims containing different or more general teachings described herein, or that the “particular” aspects are somehow limited in some way other than the inherent meanings of the language literally used therein.

Aspect 1: A device for determining when a nasal cannula is positioned properly on a user, the device comprising:
a connecting tube configured to couple to a gas source;
a first nasal prong coupled to the connecting tube and configured to insert into a first nostril of a user, the first nasal prong comprising a first nasal prong electrode;
a second nasal prong coupled to the connecting tube and configured to insert into a second nostril of the user, the second nasal prong comprising a second nasal prong electrode;
a first resistor having a predetermined, known resistance value electrically coupled to the first nasal prong electrode and the second nasal prong electrode, wherein the first nasal prong electrode and the second nasal prong electrode form a second resistor having a variable resistance value that changes depending on contact between the first and second nasal prong electrodes and skin within the first and second nostrils of the user;
a voltage source configured to introduce an input voltage across the first resistor and the second resistor; and
a voltage sensing component configured to detect an output voltage between the first resistor and the second resistor.

Aspect 2: The device according to aspect 1, wherein each of the first nasal prong and the second nasal prong comprises a proximal end coupled to the connecting tube and a distal free end configured to insert into the respective nostril, wherein the first nasal prong electrode is positioned adjacent the distal end of the first nasal prong and the second nasal prong electrode is positioned adjacent the distal end of the second nasal prong.

Aspect 3: The device according to aspect 1 or aspect 2, wherein the voltage source may comprise at least one of a microcontroller GPIO pin, battery, regulator, or digital-to-analog converter.

Aspect 4: The device according to any of the preceding aspects, wherein the voltage sensing component is at least one of an analog-to-digital converter, amplifier, voltmeter, or multimeter.

Aspect 5: The device according to any of the preceding aspects, wherein the resistance value is a value from 1 M ohm to 10 M ohm.

Aspect 6: A system comprises the device according to any of the preceding aspects and a computing device, wherein the computing device is configure to:
cause the voltage source to introduce the input voltage; and
compare the output voltage to a threshold value to determine whether the first nasal prong is positioned in the first nostril and the second nasal prong is positioned in the second nostril.

Aspect 7: The system of aspect 6, wherein the computing device is further configured to activate an alarm system of the device when the first nasal prong is determined to not be positioned in the first nostril and the second nasal prong is determined to not be positioned in the second nostril.

Aspect 8: The system of aspect 6 or aspect 7, wherein the computing device if further configured to cause actuators of the device to prevent the gas source from supplying a gas to the first nasal prong and the second nasal prong when the first nasal prong is determined to not be positioned in the first nostril and the second nasal prong is determined to not be positioned in the second nostril.

Aspect 9: A device for monitoring flow of gas from a gas source to a patient, said device comprising:
a housing;
a microcontroller positioned within the housing; and
wherein the microcontroller is configured to communicate with at least one flex sensor and at least one nasal prong electrode, wherein the microcontroller is configured to:
   receive data from at least one of at least one flex sensor or at least one nasal prong electrode;
   determine, based on the received data, whether the nasal cannula is not properly positioned; and
   cause an alarm to be triggered when the microcontroller determines the nasal cannula is not properly positioned.

Aspect 10: The device according to aspect 9, wherein the microcontroller is further configured to determine, based on the received data of at least one flex sensor, a flex level, wherein whether the nasal cannula is not properly positioned is based on the flex level.

Aspect 11: The device according to aspect 9 or aspect 10, wherein the microcontroller is further configured to determine, based on the received data of the at least one nasal prong electrode, at least one of a skin resistance level or a voltage output, wherein whether the nasal cannula is not properly positioned is based on at least the skin resistance level or the voltage output.

Aspect 12: The device according to any of aspects 9-11, wherein the microcontroller comprises a first resistor having a predetermined, known resistance value, wherein the microcontroller is further configured to introduce a voltage input across the resistor and the at least one nasal prong electrode, wherein the voltage output is based on voltage input, the resistor, and the at least one nasal prong electrode.

Aspect 13: The device according to any of aspects aspect 9-12 further comprising an actuator communicatively coupled to the microcontroller, wherein the actuator is configured to prevent flow of the gas from the gas source to the patient when the microcontroller determines the nasal cannula is not properly positioned.

Aspect 14: A system comprises the device according to aspect 12 and a computing device, wherein the computing device is configure to:
cause the microcontroller to introduce the voltage input across the resistor and the at least one nasal prong electrode; and
compare the output voltage to a threshold value to determine whether the first nasal prong is positioned in the first nostril and the second nasal prong is positioned in the second nostril.

Aspect 15: The system according to aspect 14, wherein the computing device is further configured to cause the microcontroller to trigger the alarm based on the comparison of the output voltage to the threshold value.

Aspect 16: A method for determining when a nasal cannula is positioned properly on a user, the method comprising:
introducing an input voltage across a first resistor having a predetermined, known resistance value, a first nasal prong electrode of a nasal cannula configured to be positioned within a first nostril of the user, and a second nasal prong electrode of the nasal cannula configured to be positioned within a second nostril of the user, wherein the first nasal prong electrode and the second nasal prong electrode form a second resistor having a variable resistance value that changes depending on contact between the first and second nasal prong electrodes and skin within the first and second nostrils of the user; and
determining an output voltage between the first resistor and the second resistor.

Aspect 17: The method according to aspect 16 further comprising determining the output voltage when the first nasal prong electrode is positioned within the first nostril of the user and the second nasal prong electrode is positioned within the second nostril of the user.

Aspect 18: The method according to aspect 17 further comprising comparing the output voltage to the determine output voltage when the first nasal prong electrode is positioned within the first nostril of the user and the second nasal prong electrode is positioned within the second nostril of the user.

Aspect 19: The method according to any of aspects 16-18 further comprising comparing the output voltage to a threshold value.

Aspect 20: The method according to any of aspects 16-18 further comprising activating an alarm when:
the output voltage is greater than a maximum threshold value,
the output voltage is less than a minimum threshold value, or
the output voltage is offset at least a predetermined value from the output voltage is positioned within a first nostril of a user and the second nasal prong electrode is positioned within a second nostril of the user.

What is claimed is:

1. A device for determining when a nasal cannula is positioned properly on a user, the device comprising:
a connecting tube configured to couple to a gas source;
a first nasal prong coupled to the connecting tube and configured to insert into a first nostril of a user, the first nasal prong comprising a first nasal prong electrode;
a second nasal prong coupled to the connecting tube and configured to insert into a second nostril of the user, the second nasal prong comprising a second nasal prong electrode;
a first resistor having a predetermined, known resistance value electrically coupled to the first nasal prong electrode and the second nasal prong electrode, wherein the first nasal prong electrode and the second nasal prong electrode form a second resistor having a variable resistance value that changes depending on contact between the first and second nasal prong electrodes and skin within the first and second nostrils of the user;
a voltage source configured to introduce an input voltage across the first resistor and the second resistor; and
a voltage sensing component configured to detect an output voltage between the first resistor and the second resistor.

2. The device according to claim 1, wherein each of the first nasal prong and the second nasal prong comprises a proximal end coupled to the connecting tube and a distal free end configured to insert into the respective nostril, wherein the first nasal prong electrode is positioned adjacent the distal end of the first nasal prong and the second nasal prong electrode is positioned adjacent the distal end of the second nasal prong.

3. The device according to claim 1, wherein the voltage source may comprise at least one of a microcontroller GPIO pin, battery, regulator, or digital-to-analog converter.

4. The device according to claim 1, wherein the voltage sensing component is at least one of an analog-to-digital converter, amplifier, voltmeter, or multimeter.

5. The device according to claim 1, wherein the resistance value is a value from 1 M ohm to 10 M ohm.

6. A system comprises the device according to claim 1 and a computing device, wherein the computing device is configure to:
cause the voltage source to introduce the input voltage; and
compare the output voltage to a threshold value to determine whether the first nasal prong is positioned in the first nostril and the second nasal prong is positioned in the second nostril.

7. The system of claim 6, wherein the computing device is further configured to activate an alarm system of the device when the first nasal prong is determined to not be positioned in the first nostril and the second nasal prong is determined to not be positioned in the second nostril.

8. The system of claim 6, wherein the computing device if further configured to cause actuators of the device to prevent the gas source from supplying a gas to the first nasal prong and the second nasal prong when the first nasal prong is determined to not be positioned in the first nostril and the second nasal prong is determined to not be positioned in the second nostril.

9. A device for monitoring flow of gas from a gas source to a patient, the device comprising:
a housing;
a microcontroller positioned within the housing; and
wherein the microcontroller is configured to communicate with at least one flex sensor and at least one nasal prong electrode, wherein the microcontroller is configured to:
receive data from at least one of at least one flex sensor or at least one nasal prong electrode;
determine, based on the received data, whether the nasal cannula is not properly positioned; and
cause an alarm to be triggered when the microcontroller determines the nasal cannula is not properly positioned.

10. The device according to claim 9, wherein the microcontroller is further configured to determine, based on the received data of at least one flex sensor, a flex level, wherein whether the nasal cannula is not properly positioned is based on the flex level.

11. The device according to claim 9, wherein the microcontroller is further configured to determine, based on the received data of the at least one nasal prong electrode, at least one of a skin resistance level or a voltage output, wherein whether the nasal cannula is not properly positioned is based on at least the skin resistance level or the voltage output.

12. The device according to claim 9, wherein the microcontroller comprises a first resistor having a predetermined, known resistance value, wherein the microcontroller is further configured to introduce a voltage input across the first resistor and the at least one nasal prong electrode, wherein the voltage output is based on the voltage input, the first resistor, and the at least one nasal prong electrode.

13. The device according to claim 9 further comprising an actuator communicatively coupled to the microcontroller, wherein the actuator is configured to prevent flow of the gas from the gas source to the patient when the microcontroller determines the nasal cannula is not properly positioned.

14. A system comprises the device according to claim 12 and a computing device, wherein the computing device is configure to:
cause the microcontroller to introduce the voltage input across the resistor and the at least one nasal prong electrode; and
compare the output voltage to a threshold value to determine whether the first nasal prong is positioned in the first nostril and the second nasal prong is positioned in the second nostril.

15. The system according to claim 14, wherein the computing device is further configured to cause the microcontroller to trigger the alarm based on the comparison of the output voltage to the threshold value.

16. A method for determining when a nasal cannula is positioned properly on a user, the method comprising:
introducing an input voltage across a first resistor having a predetermined, known resistance value, a first nasal prong electrode of a nasal cannula configured to be positioned within a first nostril of the user, and a second nasal prong electrode of the nasal cannula configured to be positioned within a second nostril of the user, wherein the first nasal prong electrode and the second nasal prong electrode form a second resistor having a variable resistance value that changes depending on contact between the first and second nasal prong electrodes and skin within the first and second nostrils of the user; and
determining an output voltage between the first resistor and the second resistor.

17. The method according to claim 16 further comprising determining the output voltage when the first nasal prong electrode is positioned within the first nostril of the user and the second nasal prong electrode is positioned within the second nostril of the user.

18. The method according to claim 17 further comprising comparing the output voltage to the determine output voltage when the first nasal prong electrode is positioned within the first nostril of the user and the second nasal prong electrode is positioned within the second nostril of the user.

19. The method according to claim 16 further comprising comparing the output voltage to a threshold value.

20. The method according to claim 16 further comprising activating an alarm when:
the output voltage is greater than a maximum threshold value,
the output voltage is less than a minimum threshold value, or
the output voltage is offset at least a predetermined value from the output voltage is positioned within a first nostril of a user and the second nasal prong electrode is positioned within a second nostril of the user.
